(19) United States
(12) Patent Application Publication
(10) Pub. No.: US 2026/0194402 A1
(43) Pub. Date: Jul. 9, 2026

(54) THREE-IN-ONE THERMOMETER

(71) Applicant: Shenzhen Medesigner Technology Co., LTD, Shenzhen (CN)

(72) Inventor: Min Zhu, Shenzhen (CN)

(21) Appl. No.: 19/554,602

(22) Filed: Mar. 2, 2026

(51) Int. Cl. G01K 13/20; A61B 5/0205; G01K 1/02

(52) U.S. Cl. CPC G01K 13/20; A61B 5/02055; G01K 1/02; G01K 2215/00

(57) ABSTRACT

A three-in-one thermometer includes a housing, a control board disposed within the housing, and a sensor assembly electrically connected to the control board. The sensor assembly is integrated at an insertion portion at one end of the housing, enabling synchronous measurement of body temperature, blood oxygen saturation, and heart rate during a single insertion. By integrating multiple sensors at a front end of the insertion portion and processing signals through a single control board, the thermometer achieves physical and functional integration of multiple physiological measurements. A detachable housing facilitates cleaning, while a sensor fixing bracket, a metal cap window, sealing rings, and waterproof plugs ensure accurate positioning, reliable measurement, and safe operation.

### TECHNICAL FIELD

The present disclosure relates to the field of electronic measurement devices, and more particularly to a three-in-one thermometer.

### BACKGROUND

In veterinary medicine and home health monitoring, body temperature, blood oxygen saturation, and heart rate are critical physiological parameters for evaluating vital signs. Conventionally, these parameters are measured using separate devices, such as electronic thermometers, pulse oximeters, and heart rate monitors. Such devices are independent from one another and require multiple measurement procedures.

Existing solutions require sequential use of different devices, resulting in cumbersome and time-consuming operations. It is not possible to rapidly obtain comprehensive physiological parameters in a single operation, which increases stress responses in animals and is disadvantageous for efficient assessment, particularly in emergency situations.

Accordingly, there is a need for a measurement device capable of synchronously acquiring multiple physiological parameters in a single operation.

### SUMMARY

### Technical Problem

The present disclosure aims to overcome the deficiencies of the prior art by providing a three-in-one thermometer capable of synchronously measuring body temperature, blood oxygen saturation, and heart rate during a single insertion.

### Technical Solution

To achieve the above objective, the present disclosure provides a three-in-one thermometer comprising a housing, a control board disposed within the housing, and a sensor assembly electrically connected to the control board. The sensor assembly is integrated at an insertion portion formed at one end of the housing, such that body temperature, blood oxygen saturation, and heart rate data can be synchronously acquired during a single insertion into a measurement site.

### Beneficial Effects

Compared with the prior art, the three-in-one thermometer of the present disclosure enables synchronous acquisition of multiple vital signs through a single insertion, significantly improving measurement efficiency and convenience. The detachable housing structure facilitates cleaning and maintenance. Accurate sensor positioning and sealing structures ensure reliable measurements and safe operation.

### BRIEF DESCRIPTION OF DRAWINGS

FIG. 1 is a schematic diagram showing that a housing structure of the present disclosure is provided.

FIG. 2 is a schematic diagram showing that a battery arrangement is provided in the present disclosure.

FIG. 3 is a schematic diagram showing that a bracket structure of the present disclosure is provided.

FIG. 4 is a schematic diagram showing that another housing structure of the present disclosure is provided.

FIG. 5 is a schematic diagram showing that a cross-sectional structure of an insertion portion is provided.

FIG. 6 is a schematic diagram showing that a cross-sectional structure of the housing is provided.

### DETAILED DESCRIPTION OF EMBODIMENTS

The present disclosure will be described in further detail below with reference to the accompanying drawings and specific embodiments. It should be understood that the embodiments described herein are merely exemplary embodiments of the present disclosure and are not intended to limit the scope of the invention. Based on the embodiments disclosed herein, various modifications or equivalent substitutions made by those skilled in the art without departing from the spirit of the invention shall fall within the scope of protection of the present disclosure.

### EMBODIMENT

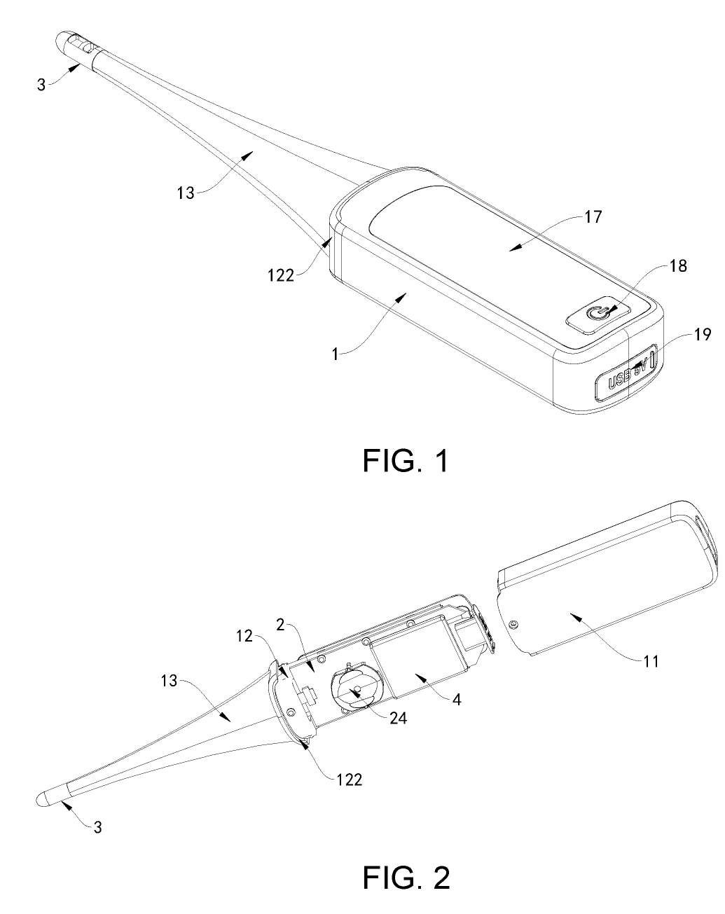

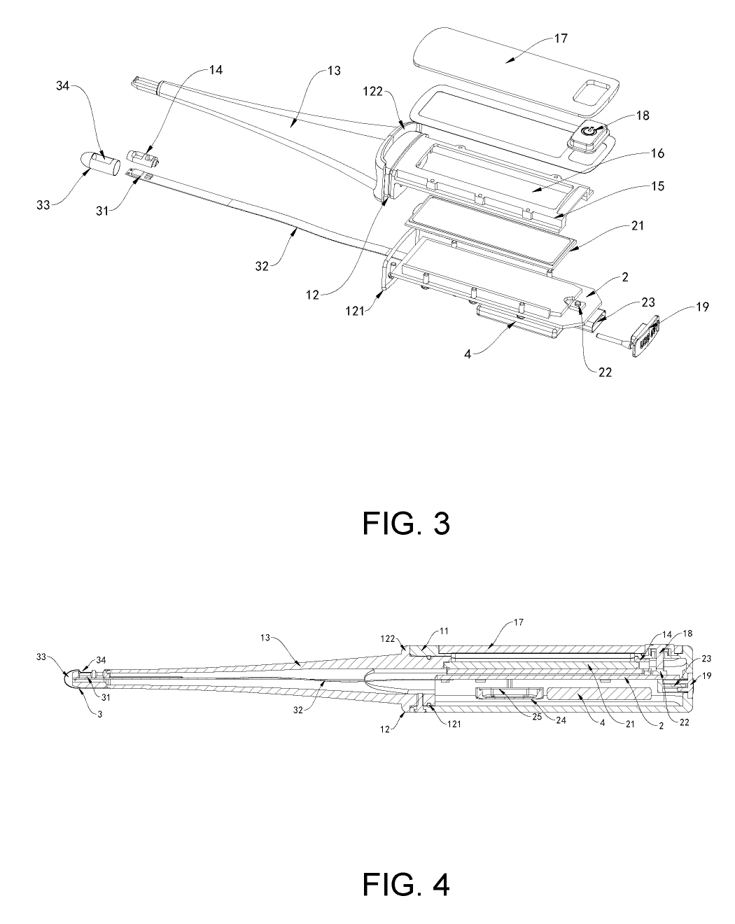

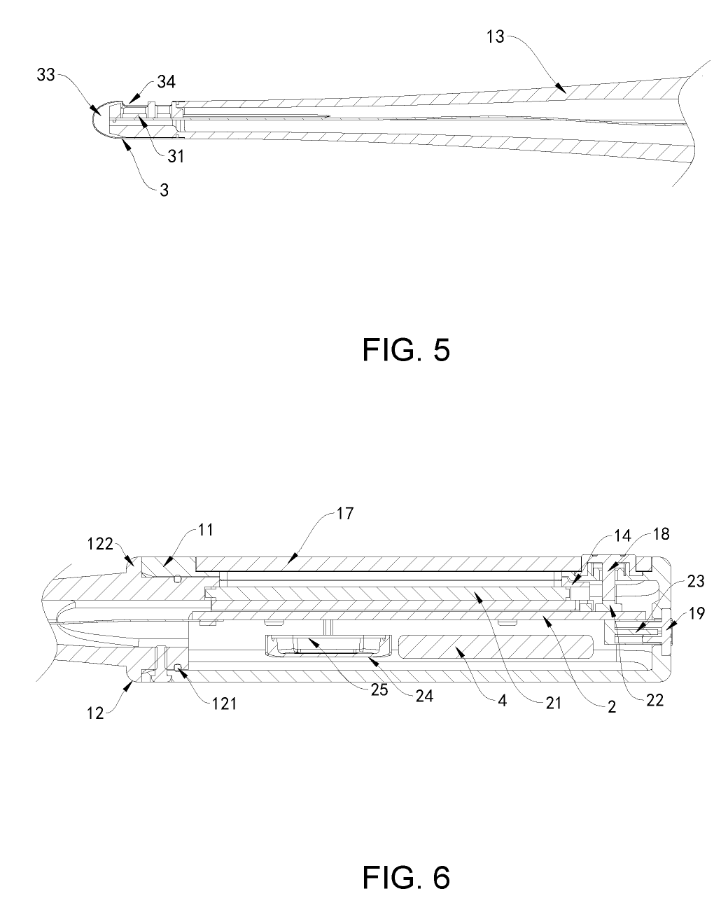

As shown in FIGS. 1-6, a three-in-one thermometer according to one embodiment of the present disclosure comprises a housing 1, a control board 2 disposed inside the housing 1, and a sensor assembly 3 electrically connected to the control board 2. The sensor assembly 3 is integrated at an insertion portion 13 formed at one end of the housing 1 and is configured to synchronously acquire body temperature, blood oxygen saturation, and heart rate data during a single insertion into a measurement site.

Specifically, sensing functional units that were conventionally separated—namely, a temperature sensing unit, a blood oxygen sensing unit, and a heart rate sensing unit—are physically integrated at a front end of the device configured for insertion into a body cavity, i.e., the insertion portion 13. The sensing signals are jointly controlled and processed by a single control board 2, thereby achieving a functional breakthrough in which three key physiological parameters are synchronously measured through a single insertion.

This configuration significantly improves measurement efficiency, avoids repeated insertion operations that may cause stress or discomfort to animals, and is particularly suitable for veterinary home care and clinical scenarios requiring rapid and comprehensive acquisition of vital signs.

As shown in FIGS. 1-6, in some embodiments, the housing 1 comprises a lower housing 11 and a bracket 12 detachably mounted to one end of the lower housing 11. An end of the bracket 12 distal from the lower housing 11 forms the insertion portion 13 with a gradually reduced diameter. The control board 2 is disposed inside the lower housing 11.

The lower housing 11 serves as a main body for accommodating core electronic components, such as the control board 2, while the bracket 12 specifically forms the front insertion portion 13. The lower housing 11 and the bracket 12 are detachably connected, for example, via a screw fastened from a bottom of the lower housing 11 into a mounting portion 15 of the bracket 12.

Such a separable structural design facilitates assembly, maintenance, and cleaning. The insertion portion 13, which is more susceptible to contamination or wear, can be independently detached for thorough cleaning or replacement, thereby ensuring hygiene and reducing maintenance costs. In addition, the modular configuration improves manufacturing and assembly efficiency.

As shown in FIGS. 1-6, in some embodiments, the sensor assembly 3 comprises a blood oxygen and heart rate sensor module 31. The blood oxygen and heart rate sensor module 31 is electrically connected to the control board 2 via an electrical connection member 32 extending into the insertion portion 13.

The blood oxygen and heart rate sensor module 31 may comprise a light-emitting element, such as a light-emitting diode (LED), and a photodetector. The electrical connection member 32 may be a flexible printed circuit, a wire harness, or a micro connector extending through an internal cavity of the bracket 12 to establish signal and power transmission with the control board 2.

This configuration allows the optical sensing unit to be positioned at the limited space at the front end of the insertion portion 13, enabling direct contact with a measurement site, such as rectal mucosa. As a result, high-quality photoplethysmography (PPG) signals can be acquired, providing a reliable basis for accurate calculation of blood oxygen saturation and heart rate.

As shown in FIGS. 1-6, in some embodiments, the sensor assembly 3 further comprises a metal cap 33 sleeved over a front end of the insertion portion 13. The metal cap 33 is integrally formed with a temperature sensor to constitute a one-piece temperature sensing head.

The metal cap 33 may be made of stainless steel or another thermally conductive material. A temperature sensing element, such as a thermistor, is fixed inside the metal cap 33 via a thermally conductive insulating material, thereby forming a robust and fast-response temperature sensing structure.

The integrated design ensures excellent thermal conduction between the temperature sensor and the measured tissue, shortens thermal equilibrium time, and improves temperature measurement accuracy. The metal cap 33 also provides mechanical protection for the internal sensor and features a smooth surface that is easy to clean and disinfect.

As shown in FIGS. 1-6, in some embodiments, one end of the insertion portion 13 is further provided with a sensor fixing bracket 14 configured to fix the blood oxygen and heart rate sensor module 31. The sensor fixing bracket 14 is nested within an interior of the metal cap 33. The metal cap 33 is provided with a window 34 exposing a detection surface of the blood oxygen and heart rate sensor module 31.

The sensor fixing bracket 14 is an independent internal support structure that precisely positions and secures the blood oxygen and heart rate sensor module 31. The sensor fixing bracket 14 ensures that the detection surface of the sensor module 31 is accurately aligned with the window 34 formed on the metal cap 33.

The window 34 may be sealed with a light-transmissive material, thereby protecting the internal sensor while allowing effective emission and reception of optical signals. This configuration prevents displacement of the sensor module 31, ensures stable optical alignment, and improves consistency and reliability of measurement results.

As shown in FIGS. 1-6, in some embodiments, an end of the bracket 12 distal from the insertion portion 13 is provided with a mounting portion 15 extending into the lower housing 11. The control board 2 is integrated with a display screen 21. A top of the display screen 21 is embedded within the mounting portion 15. A display window 16 is formed at a top of the mounting portion 15.

The mounting portion 15 defines a cavity or frame configured to receive and protect the display screen 21. After the bracket 12 is mounted to the lower housing 11, the display screen 21 is aligned with and visible through the display window 16. This structure utilizes the internal space of the mounting portion 15 to achieve a compact and integrated layout, while providing lateral support and protection for the display screen 21.

As shown in FIGS. 1-6, in some embodiments, a panel 17 corresponding to the display window 16 is embedded at a top of the lower housing 11. The control board 2 is integrated with control keys 22. Buttons 18 corresponding to the control keys 22 are provided on the lower housing 11.

The panel 17 serves as an exterior cover and protective component and is aligned with the display window 16. The buttons 18 are aligned in a vertical direction with the control keys 22 on the control board 2. When a user presses the buttons 18, corresponding control operations such as power-on, power-off, or measurement initiation are triggered.

This configuration provides an intuitive and reliable human–machine interface, improves user experience, and enhances overall appearance and durability of the device.

As shown in FIGS. 1-6, in some embodiments, a battery 4 electrically connected to the control board 2 is disposed inside the lower housing 11. A charging interface 23 is integrated at a bottom of the control board 2. A waterproof plug 19 corresponding to the charging interface 23 is movably fitted at one end of the lower housing 11.

The battery 4 supplies power to the entire device. The charging interface 23 may be a Micro-USB interface, a Type-C interface, or another suitable charging interface. The waterproof plug 19 may be made of elastic rubber or silicone and is configured to tightly seal the charging interface opening when the device is not being charged.

The waterproof plug 19 effectively prevents liquid or dust from entering the housing through the charging interface, thereby improving durability and safety, particularly in veterinary use environments.

As shown in FIGS. 1-6, in some embodiments, a buzzer 25 is integrated at a bottom of the control board 2 via a buzzer cover 24.

The buzzer 25 is configured to provide audible prompts, such as indicating completion of measurement or low battery status. The buzzer cover 24 secures and protects the buzzer 25 and may also function as a resonance cavity to enhance sound output.

As shown in FIGS. 1-6, in some embodiments, a sealing ring 121 is provided at a connection between the bracket 12 and the lower housing 11. The connection forms a radially protruding flange 122. An outer contour of the flange 122 smoothly transitions to an end of the lower housing 11.

The sealing ring 121 prevents liquid ingress through an assembly gap between the bracket 12 and the lower housing 11, thereby protecting internal electronic components. The smooth transition of the flange 122 improves ergonomics, appearance, and ease of cleaning.

During use, when the insertion portion 13 integrated with the metal cap 33 and the blood oxygen and heart rate sensor module 31 is inserted into an animal’s rectum, the metal cap 33 directly contacts the intestinal wall to rapidly conduct heat and accurately measure body temperature. Simultaneously, optical signals emitted by the blood oxygen and heart rate sensor module 31 are directed toward mucosal tissue through the window 34. Light reflected after absorption by blood is received by a photodetector to acquire raw photoplethysmography (PPG) signals.

The control board 2 processes electrical signals from the temperature sensor and the optical sensor module in real time. Embedded algorithms calculate body temperature, blood oxygen saturation, and heart rate values, which are then synchronously displayed on the display screen 21, thereby completing efficient acquisition of three physiological parameters through a single insertion.

In summary, by integrating a body temperature sensor, a blood oxygen sensor, and a heart rate sensor at a front end of the same insertion portion 13 and processing the acquired signals through a single control board 2, the present disclosure achieves physical and functional integration of body temperature, blood oxygen saturation, and heart rate measurements. All measurements can be synchronously completed through a single insertion, thereby significantly improving the efficiency and convenience of veterinary diagnosis and treatment. The separable housing 1 design enables the insertion portion 13 containing the sensor assembly 3 to be easily detached for cleaning, thereby ensuring hygienic safety. In addition, precise positioning achieved by the cooperation of the sensor fixing bracket 14 and the window 34 of the metal cap 33 ensures reliability of the measurements. The entire device is provided with necessary protective structures by means of the sealing ring 121 and the waterproof plug 19, making the device safer and more convenient to use.

Finally, it should be noted that the foregoing descriptions are merely preferred embodiments of the present disclosure and are not intended to limit the scope of the invention. Although the present disclosure has been described in detail with reference to the foregoing embodiments, those skilled in the art may still make modifications to the technical solutions described in the embodiments or make equivalent substitutions for certain technical features thereof. Any modifications, equivalent substitutions, or improvements made within the spirit and principles of the present disclosure shall fall within the scope of protection of the present disclosure.

1. A three-in-one thermometer, comprising:
a housing;
a control board disposed within the housing; and
a sensor assembly electrically connected to the control board,
the sensor assembly is integrated at an insertion portion formed at one end of the housing and is configured to synchronously acquire body temperature, blood oxygen saturation, and heart rate data during a single insertion into a measurement site.

2. The three-in-one thermometer according to claim 1,wherein the housing comprises a lower housing and a bracket detachably mounted to one end of the lower housing, an end of the bracket distal from the lower housing forms the insertion portion with a gradually reduced diameter, and the control board is disposed within the lower housing.

3. The three-in-one thermometer according to claim 1, wherein the sensor assembly comprises a blood oxygen and heart rate sensor module, and the blood oxygen and heart rate sensor module is electrically connected to the control board via an electrical connection member extending into the insertion portion.

4. The three-in-one thermometer according to claim 1, wherein the sensor assembly further comprises a metal cap sleeved over a front end of the insertion portion, and the metal cap is integrally formed with a temperature sensor to constitute a one-piece temperature sensing head.

5. The three-in-one thermometer according to claim 1, wherein one end of the insertion portion is provided with a sensor fixing bracket configured to fix the blood oxygen and heart rate sensor module, the sensor fixing bracket is nested within an interior of the metal cap, and the metal cap is provided with a window exposing a detection surface of the blood oxygen and heart rate sensor module.

6. The three-in-one thermometer according to claim 2, wherein an end of the bracket distal from the insertion portion is provided with a mounting portion extending into the lower housing,
the control board is integrated with a display screen, a top of the display screen is embedded within the mounting portion, and a display window is formed at a top of the mounting portion.

7. The three-in-one thermometer according to claim 6, wherein a panel corresponding to the display window is embedded at a top of the lower housing, the control board is integrated with control keys, and buttons corresponding to the control keys are provided on the lower housing.

8. The three-in-one thermometer according to claim 2, wherein a battery electrically connected to the control board is disposed within the lower housing, a charging interface is integrated at a bottom of the control board, and a waterproof plug corresponding to the charging interface is movably fitted at one end of the lower housing.

9. The three-in-one thermometer according to claim 1, wherein a buzzer is integrated at a bottom of the control board via a buzzer cover.

10. The three-in-one thermometer according to claim 2, wherein a sealing ring is provided at a connection between the bracket and the lower housing, the connection forms a radially protruding flange, and an outer contour of the flange smoothly transitions to an end of the lower housing.
